(19) United States
(12) Patent Application Publication
(10) Pub. No.: US 2020/0138383 A1
(43) Pub. Date: May 7, 2020

(54) MEDICAL INFORMATION PROCESSING APPARATUS AND MEDICAL INFORMATION PROCESSING METHOD

(71) Applicant: CANON MEDICAL SYSTEMS CORPORATION, Otawara-shi (JP)

(72) Inventors: Kazumasa NORO, Shioyagun (JP); Kazuki UTSUNOMIYA, Nasushiobara (JP); Yudai Yamazaki, Otawara (JP)

(73) Assignee: CANON MEDICAL SYSTEMS CORPORATION, Otawara-shi (JP)

(21) Appl. No.: 16/659,620

(22) Filed: Oct. 22, 2019

(30) Foreign Application Priority Data
Oct. 25, 2018 (JP) 2018-200529

(51) Int. Cl. A61B 5/00; G16H 50/20

(52) U.S. Cl. CPC A61B 5/743; G16H 50/20; A61B 5/7425; A61B 5/7445; A61B 5/7435

(57) ABSTRACT

A medical information processing apparatus according to an embodiment includes a specifying unit, an obtaining unit, a determining unit, and a display controlling unit. The specifying unit specifies principal data from among a plurality of types of diagnosis/treatment data. The obtaining unit obtains peripheral data that is diagnosis/treatment data having a high degree of relevance to the principal data, by using the principal data as a reference. The determining unit determines a screen configuration indicating positional arrangements of the principal data and the peripheral data to be used at the time of displaying the principal data and the peripheral data, in accordance with an attribute of the peripheral data with respect to the principal data. The display controlling unit displays the principal data and the peripheral data on a display screen of a display by using the screen configuration.

### CROSS-REFERENCE TO RELATED APPLICATIONS

This application is based upon and claims the benefit of priority from Japanese Patent Application No. 2018-200529, filed on Oct. 25, 2018; the entire contents of which are incorporated herein by reference.

### FIELD

Embodiments described herein relate generally to a medical information processing apparatus and a medical information processing method.

### BACKGROUND

In recent years, as the medical technology becomes more advanced, the types and the number of pieces of diagnosis and/or treatment data (hereinafter, simply “diagnosis/treatment data”) acquired in diagnosis and treatment processes on a daily basis are increasing. Also, methods used by medical doctors for determining diagnoses and treatment plans with reference to diagnosis/treatment data are becoming more complicated. For these reasons, in the actual medical workplaces, systems are known by which various types of diagnosis/treatment data necessary when a medical doctor evaluates diagnoses and treatment plans are displayed on a single screen.

### BRIEF DESCRIPTION OF THE DRAWINGS

FIG. 1 is a diagram illustrating an exemplary configuration of a medical information processing apparatus according to a first embodiment;

FIGS. 2 and 3 are drawings for explaining a specifying function according to the first embodiment;

FIGS. 4 to 7 are drawings for explaining an obtaining function according to the first embodiment;

FIGS. 8 to 16 are drawings for explaining a determining function according to the first embodiment;

FIGS. 17 and 18 are drawings for explaining a display controlling function according to the first embodiment;

FIG. 19 is a flowchart illustrating a processing procedure of processes realized by processing functions of processing circuitry according to the first embodiment;

FIG. 20 is a flowchart illustrating a detailed processing procedure of the process realized by a determining function at step S13 in FIG. 19;

FIGS. 21 and 22 are drawings for explaining a determining function according to a second embodiment; and

FIG. 23 is a drawing for explaining an obtaining function according to a third embodiment.

### DETAILED DESCRIPTION

A medical information processing apparatus according to an embodiment includes a specifying unit, an obtaining unit, a determining unit, and a display controlling unit. The specifying unit is configured to specify principal data from among a plurality of types of diagnosis/treatment data. The obtaining unit is configured to obtain peripheral data that is diagnosis/treatment data having a high degree of relevance to the principal data, by using the principal data as a reference. The determining unit is configured to determine a screen configuration indicating positional arrangements of the principal data and the peripheral data to be used at the time of displaying the principal data and the peripheral data, in accordance with an attribute of the peripheral data with respect to the principal data. The display controlling unit is configured to display the principal data and the peripheral data on a display screen of a display by using the screen configuration.

Exemplary embodiments of a medical information processing apparatus and a medical information processing method will be explained in detail, with reference to the accompanying drawings.

### First Embodiment

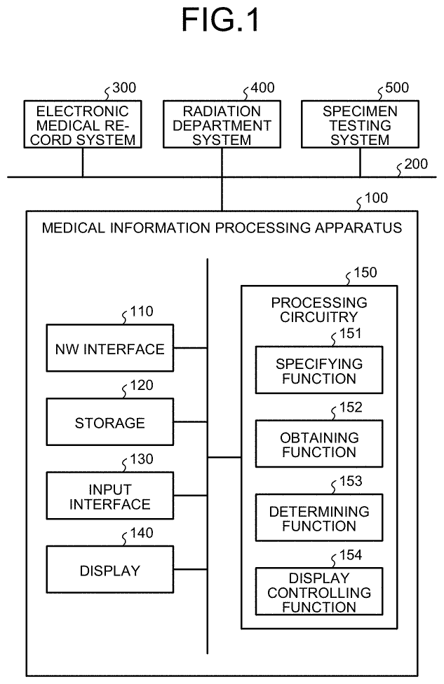

FIG. 1 is a diagram illustrating an exemplary configuration of a medical information processing apparatus according to a first embodiment.

For example, as illustrated in FIG. 1, a medical information processing apparatus 100 according to the present embodiment is communicably connected to an electronic medical record system 300, a radiation department system 400, a specimen testing system 500, and the like, via a network 200. For example, the medical information processing apparatus 100 and the systems are installed in a hospital or the like and are connected to one another by the network 200 such as an intra-hospital Local Area Network (LAN).

The electronic medical record system 300 is configured to generate diagnosis/treatment data related to prescriptions and nursing records provided for an examined subject (hereinafter “patient”) and to store the generated data into a storage within the system. Further, the electronic medical record system 300 is configured to transmit any of the diagnosis/treatment data stored in the storage to the medical information processing apparatus 100, in response to a request from the medical information processing apparatus 100.

The radiation department system 400 is configured to generate diagnosis/treatment data related to a vital test or an image examination performed on the patient and to store the generated data into a storage within the system. In this situation, examples of the image examination include: an examination using a Computed Tomography (CT) image taken by an X-ray Computed Tomography (CT) apparatus; an examination using a Magnetic Resonance (MR) image taken by a Magnetic Resonance Imaging (MRI) apparatus; an examination using an ultrasound image taken by an ultrasound diagnosis apparatus, and an examination using an X-ray image taken by an X-ray diagnosis apparatus. For example, the radiation department system 400 includes a Picture Archiving and Communication System (PACS) or the like and is configured to store medical images such as CT images, MR images, ultrasound images, X-ray images, and the like into a database, by using a format compliant with a Digital Imaging and Communications in Medicine (DICOM) scheme. Further, the radiation department system 400 is configured to transmit any of the diagnosis/treatment data stored in the storage to the medical information processing apparatus 100 in response to a request from the medical information processing apparatus 100.

The specimen testing system 500 is configured to generate diagnosis/treatment data related to a specimen test performed on the patient and store the generated data into a storage within the system. Further, the specimen testing system 500 is configured to transmit any of the diagnosis/treatment data stored in the storage to the medical information processing apparatus 100 in response to a request from the medical information processing apparatus 100.

The medical information processing apparatus 100 is configured to obtain any of the various types of diagnosis/treatment data from the electronic medical record system 300, the radiation department system 400, and the specimen testing system 500, via the network 200 and to perform various types of information processing processes by using the obtained diagnosis/treatment data. For example, the medical information processing apparatus 100 may be realized by using a computer device such as a workstation, a personal computer, or a tablet terminal.

More specifically, the medical information processing apparatus 100 includes a network (NW) interface 110, a storage 120, an input interface 130, a display 140, and processing circuitry 150.

The NW interface 110 is connected to the processing circuitry 150 and is configured to control transfer of various types of data and communication performed between the medical information processing apparatus 100 and the systems. More specifically, the NW interface 110 is configured to receive the diagnosis/treatment data from the systems and to output the received diagnosis/treatment data to the processing circuitry 150. For example, the NW interface 110 may be realized by using a network card, a network adaptor, a Network Interface Controller (NIC), or the like.

The storage 120 is connected to the processing circuitry 150 and is configured to store therein various types of data. More specifically, the storage 120 is configured to store therein the diagnosis/treatment data received from the systems. For example, the storage 120 may be realized by using a semiconductor memory element such as a Random Access Memory (RAM) or a flash memory, or a hard disk, an optical disk, or the like. The storage 120 is an example of the storage.

The input interface 130 is connected to the processing circuitry 150 and is configured to receive input operations of various types of instructions and various types of information from an operator. More specifically, the input interface 130 is configured to convert the input operations received from the operator into electrical signals and to output the electrical signals to the processing circuitry 150. For example, the input interface 130 may be realized by using a trackball, a switch button, a mouse, a keyboard, a touchpad on which an input operation is performed by touching the operation surface thereof, a touch screen in which a display screen and a touchpad are integrally formed, a contactless input circuit using an optical sensor, an audio input circuit, and/or the like. In the present disclosure, the input interface 130 does not necessarily have to include one or more physical operation component parts such as a mouse and a keyboard. For instance, possible examples of the input interface 130 include an electrical signal processing circuitry configured to receive an electrical signal corresponding to an input operation from an external input device provided separately from the apparatus and to output the electrical signal to a controlling circuit.

The display 140 is connected to the processing circuitry 150 and is configured to display various types of information and various types of images. More specifically, the display 140 is configured to convert data of the various types of information and the various types of images sent thereto from the processing circuitry 150 into display-purpose electrical signals and to output the electrical signals. For example, the display 140 may be realized by using a liquid crystal monitor, a Cathode Ray Tube (CRT) monitor, a touch panel, or the like.

The processing circuitry 150 is configured to control constituent elements of the medical information processing apparatus 100 in response to an input operation received from the operator via the input interface 130. More specifically, the processing circuitry 150 is configured to store the diagnosis/treatment data output from the NW interface 110 into the storage 120. Further, the processing circuitry 150 is configured to read any of the diagnosis/treatment data from the storage 120 and to display the read data on the display 140.

An exemplary configuration of the medical information processing apparatus 100 according to the present embodiment has thus been explained. The medical information processing apparatus 100 structured as described above is configured to cause the various types of diagnosis/treatment data obtained from the electronic medical record system 300, the radiation department system 400, and the specimen testing system 500 to be accumulated in an integrated diagnosis/treatment database (DB) constructed by the storage 120.

Further, the medical information processing apparatus 100 has a function of displaying, on a single screen, various types of diagnosis/treatment data that are necessary when a medical doctor evaluates diagnoses and treatment plans, by using the diagnosis/treatment data accumulated in the integrated diagnosis/treatment DB. For example, the medical information processing apparatus 100 divides a display screen of the display 140 into a plurality of regions and displays mutually-different types of diagnosis/treatment data in the regions.

In this situation, in the present embodiment, the medical information processing apparatus 100 is configured so that the operator is able to compare the various types of diagnosis/treatment data with one another from multiple aspects.

More specifically, in the present embodiment, the processing circuitry 150 includes a specifying function 151, an obtaining function 152, a determining function 153, and a display controlling function 154. The specifying function 151 is an example of the specifying unit. The obtaining function 152 is an example of the obtaining unit. The determining function 153 is an example of the determining unit. The display controlling function 154 is an example of the display controlling unit.

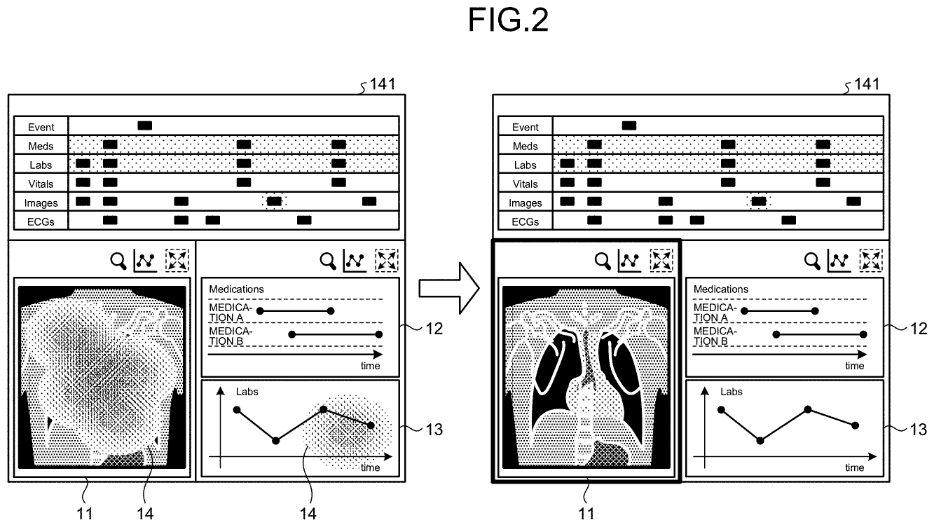

Processing functions of the processing circuitry 150 will be explained in detail below, with reference to FIGS. 2 to 22. In the present embodiment, an example will be explained in which, as the display 140, a head mounted display (HMD) or a high resolution display such as an 8K monitor is used.

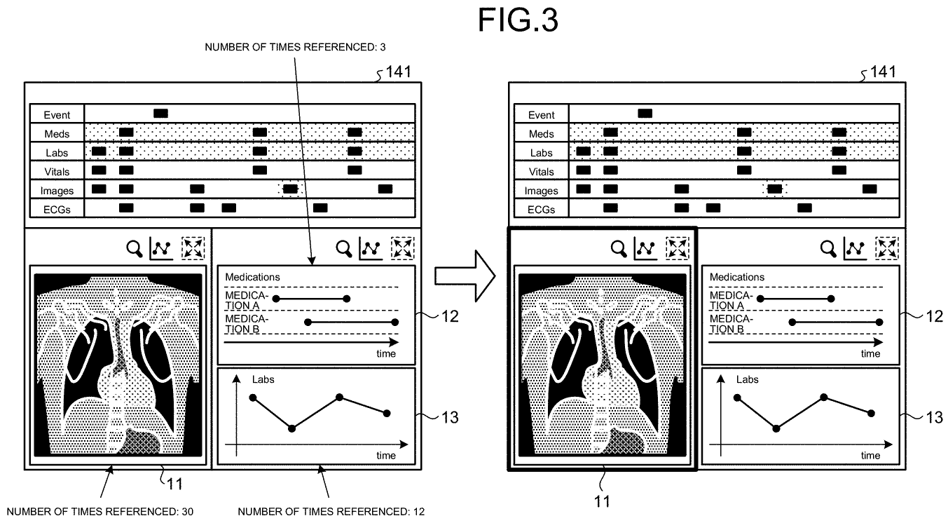

FIGS. 2 and 3 are drawings for explaining the specifying function 151 according to the first embodiment.

The specifying function 151 is configured to specify principal data from among a plurality of types of diagnosis/treatment data.

More specifically, the specifying function 151 is configured to detect diagnosis/treatment data focused on by the operator from among the plurality of types of diagnosis/treatment data being displayed on the display 140 and to specify the detected diagnosis/treatment data as the principal data.

In this situation, the specifying function 151 is configured to specify the principal data on the basis of a line of sight of the operator. For example, by using line-of-sight tracking technology such as eye tracking, the specifying function 151 is configured to successively detect gaze positions indicating the positions gazed upon by the operator on the display screen of the display 140 and to measure a time period during which a gaze position is continuously detected, for each of the regions in which the pieces of diagnosis/treatment data are displayed on the display screen. Further, when there is a region in which the gaze position is continuously detected for a predetermined time period, the specifying function 151 specifies the diagnosis/treatment data displayed in the region as the principal data.

For instance, as illustrated in FIG. 2, let us discuss an example in which image data 11, medication administration data 12, and specimen test data 13 are displayed as diagnosis/treatment data on a display screen 141 of the display 140. In that situation, for example, the specifying function 151 successively detects the gaze positions indicating the positions gazed upon by the operator on the display screen 141 and further displays heat maps 14 indicating a distribution of the detected gaze positions on the display screen 141. Further, with respect to the region displaying the image data 11, the region displaying the medication administration data 12, and the region displaying the specimen test data 13 on the display screen 141, the specifying function 151 measures a time period during which a gaze position is continuously detected. Further, in the region displaying the image data 11, for example, when a gaze position has continuously been detected for a predetermined time period, the specifying function 151 specifies the image data 11 as the principal data.

Alternatively, the specifying function 151 may specify principal data on the basis of an operation history of the operator. For example, among the pieces of diagnosis/treatment data that are currently displayed on the display screen 141 of the display 140, the specifying function 151 specifies diagnosis/treatment data that has been referenced by the same operator the largest number of times in the past, as the principal data.

For instance, let us discuss an example in which, as illustrated in FIG. 3, the image data 11, the medication administration data 12, and the specimen test data 13 are displayed as diagnosis/treatment data on the display screen 141 of the display 140. In this situation, for example, with respect to the image data 11, the medication administration data 12, and the specimen test data 13 that are currently displayed, when the number of times being referenced is 30 for the image data 11, the number of times being referenced is 3 for the medication administration data 12, and the number of times being referenced is 12 for the specimen test data 13, the specifying function 151 specifies the image data 11 as principal data, as illustrated on the right-hand side of FIG. 3.

In another example, from among the various types of diagnosis/treatment data displayed on the display screen 141, the specifying function 151 may specify a type that has been referenced by the same operator the largest number of times in the past and may specify the most recent piece of diagnosis/treatment data among the specified type of diagnosis/treatment data, as principal data. For example, when image data 21 has been referenced the largest number of times, the specifying function 151 specifies the most recent piece of image data among the image data 21 as the principal data.

In yet another example, the specifying function 151 may speculate diagnosis/treatment data to be referenced next by the operator on the basis of an operation history of the operator by using a machine learning method, to specify the speculated diagnosis/treatment data as principal data. In that situation, for example, the specifying function 151 uses a trained model generated by a machine learning process for which the operation history and the diagnosis/treatment data from the past of the operator are used as learning-purpose data. Further, the specifying function 151 exercises control so that, by inputting the operation history of the operator to the trained model, the diagnosis/treatment data to be referenced next by the operator is output.

In the present example, the situations were explained in which the specifying function 151 detects the diagnosis/treatment data focused on by the operator, on the basis of one of: the line of sight of the operator; and the operation history of the operator. However, possible embodiments are not limited to this example. For instance, the specifying function 151 may detect the diagnosis/treatment data focused on by the operator, by receiving, from the operator, an operation to designate a piece of diagnosis/treatment data from among the plurality of types of diagnosis/treatment data displayed on the display 140, via the input interface 130.

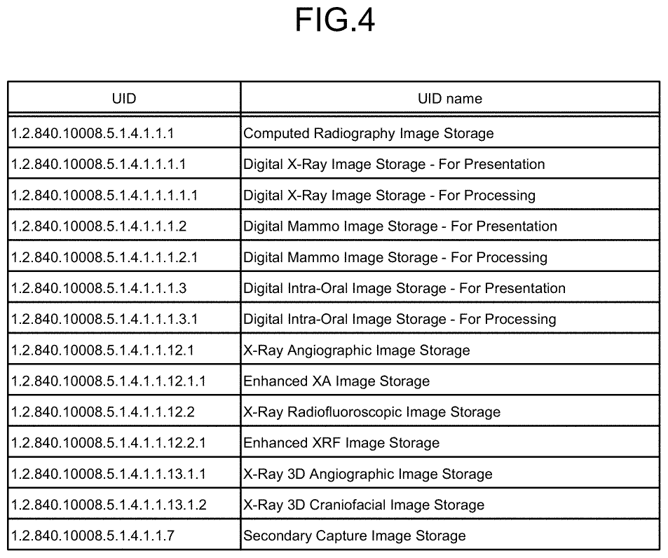

FIGS. 4 to 7 are drawings for explaining the obtaining function 152 according to the first embodiment.

By using the principal data specified by the specifying function 151 as a reference, the obtaining function 152 is configured to obtain peripheral data that is diagnosis/treatment data having a high degree of relevance to the principal data.

More specifically, the obtaining function 152 is configured to derive a degree of relevance for each piece of diagnosis/treatment data by using the principal data as a reference and to obtain a piece of diagnosis/treatment data having a high degree of relevance as the peripheral data.

In this situation, on the basis of identification information contained in the diagnosis/treatment data, the obtaining function 152 categorizes the peripheral data as one of: information of the same type as the principal data; information explaining the principal data; and information having a high possibility of being compared with the principal data. After that, the obtaining function 152 derives the degree of relevance on the basis of which of the following the peripheral data is: the information of the same type as the principal data; the information explaining the principal data; and the information having a high possibility of being compared with the principal data.

More specifically, the obtaining function 152 obtains peripheral data to be categorized as the information of the same type as the principal data, on the basis of an “SOP Class UID” in a DICOM tag (0008,0016) contained in diagnosis/treatment data.

For example, as illustrated in FIG. 4, the DICOM tag (0008,0016) is set with the “SOP Class UID” (UID) identifying a set made up of an object and a service defined by the DICOM standard and a “UID name” indicating the specifics of the set. In this situation, by referring to the “SOP Class UID” in the DICOM tag (0008,0016), it is possible to identify pieces of diagnosis/treatment data that are of the same type.

For example, the obtaining function 152 extracts the value of the “SOP Class UID” from the DICOM tag (0008,0016) contained in the principal data and obtains, from among the pieces of diagnosis/treatment data stored in the integrated diagnosis/treatment DB, a piece of diagnosis/treatment data that has the “SOP Class UID” of the same value as the extracted value. Subsequently, with respect to the obtained piece of diagnosis/treatment data, the obtaining function 152 derives a degree of relevance to the principal data on the basis of the acquisition time (the date, the time, or the like) of each of the pieces of peripheral data. In this situation, the obtaining function 152 derives the degree of relevance of each of the pieces of peripheral data in such a manner that the closer the acquisition time of a piece of peripheral data is to the acquisition time of the principal data, the higher is the degree of relevance. After that, the obtaining function 152 obtains such diagnosis/treatment data of which the derived degree of relevance is equal to or higher than a threshold value as the peripheral data from the integrated diagnosis/treatment DB and categorizes the peripheral data as the information that is of the same type as the principal data.

Further, on the basis of identification information identifying reference data written in a format of DICOM Structured Report (SR) and contained in the diagnosis/treatment data, the obtaining function 152 obtains peripheral data to be categorized as the information explaining the principal data.

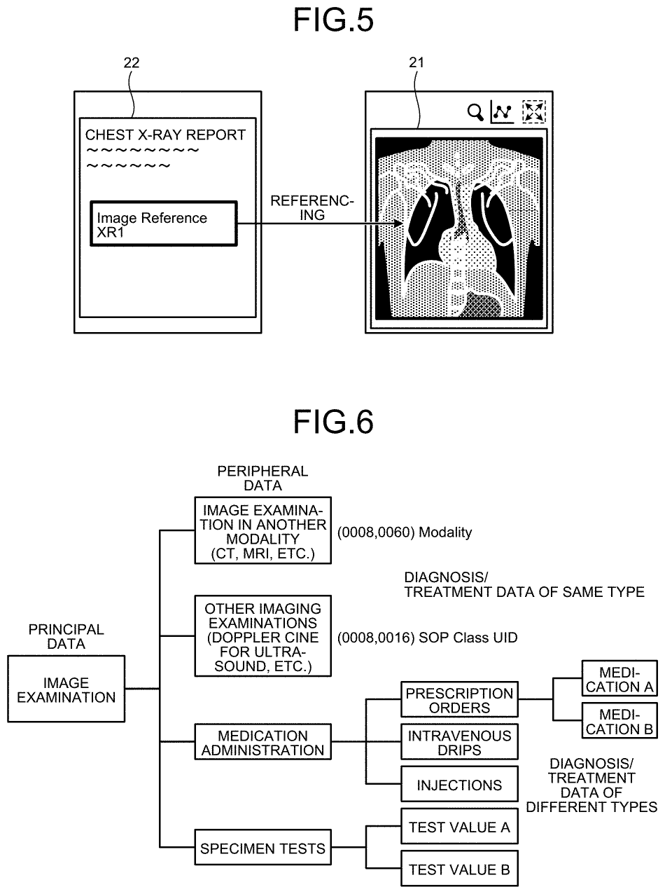

For example, as illustrated in FIG. 5, from among the diagnosis/treatment data written in the format of DICOM SR and stored in the integrated diagnosis/treatment DB, the obtaining function 152 obtains diagnosis/treatment data 22 referencing principal data 31, on the basis of information of “Image Reference” indicating reference data. Subsequently, with respect to the obtained diagnosis/treatment data 22, the obtaining function 152 derives a degree of relevance to the principal data, on the basis of a level of detail of each of the pieces of peripheral data. In this situation, the obtaining function 152 derives the degree of relevance of each of the pieces of peripheral data in such a manner that the closer the level of detail of a piece of peripheral data is to the level of detail of the principal data, the higher is the degree of relevance. After that, the obtaining function 152 obtains such diagnosis/treatment data of which the derived degree of relevance is equal to or higher than a threshold value as the peripheral data from the integrated diagnosis/treatment DB and categorizes the peripheral data as the information explaining the principal data.

Further, on the basis of relevance degree information stored in the storage 120, the obtaining function 152 obtains peripheral data to be categorized as the information having a high possibility of being compared with the principal data.

In this situation, on the basis of a DICOM tag or the like contained in the diagnosis/treatment data, a plurality of pieces of diagnosis/treatment data that can serve as pieces of peripheral data are defined in advance for each principal data.

For example, as illustrated in FIG. 6, with respect to the principal data, diagnosis/treatment data of the same type and diagnosis/treatment data of different types are defined. For example, as the diagnosis/treatment data of the same type, diagnosis/treatment data related to an image examination using another modality (e.g., an X-ray CT apparatus or an MRI apparatus) is defined, on the basis of “Modality” in the DICOM tag (0080,0060). As another example, as the diagnosis/treatment data of the same type, diagnosis/treatment data related to another imaging examination (e.g., a Doppler cine examination for an ultrasound examination) is defined on the basis of an “SOP Class UID” in the DICOM tag (0008,0016). As yet another example, diagnosis/treatment data related to medication administration and diagnosis/treatment data related to specimen tests are defined as the diagnosis/treatment data of different types. In this situation, for example, within the diagnosis/treatment data related to the medication administration, diagnosis/treatment data related to prescription orders, diagnosis/treatment data related to intravenous drips, and diagnosis/treatment data related to injections are further defined. Further, for example, within the diagnosis/treatment data related to the prescription orders, diagnosis/treatment data related to medication A and diagnosis/treatment data related to medical B are further defined.

Further, for each piece of diagnosis/treatment data defined in the manner described above, the storage 120 stores therein the relevance degree information in which the number of times the piece of diagnosis/treatment data has been browsed or operated together with the principal data is set as a degree of relevance.

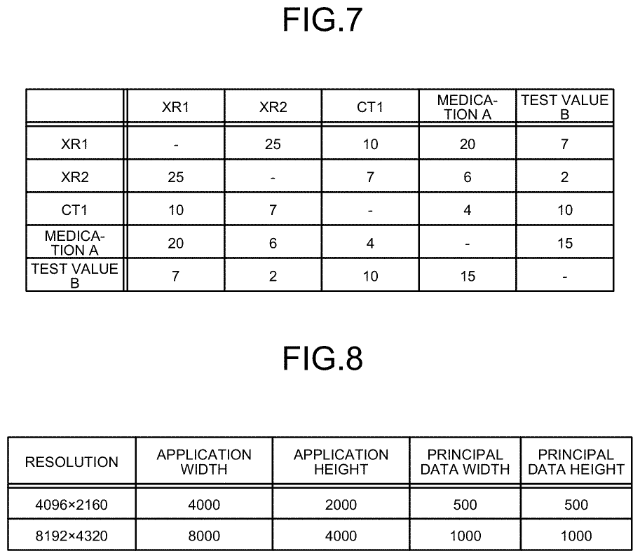

For example, as illustrated in FIG. 7, in the relevance degree information, set as the degrees of relevance are the number of times each piece of diagnosis/treatment data has been browsed or operated together with each piece of principal data, which are kept in correspondence with: pieces of identification information identifying the pieces of principal data (“XR1”, “XR2”, “CT1”, “Medication A”, and “Test Value B” on the farthest left in each of the lines of the table in FIG. 7); and pieces of identification information identifying the pieces of diagnosis/treatment data (“XR1”, “XR2”, “CT1”, “Medication A”, and “Test Value B” at the top of each of the columns of the table in FIG. 7). In this situation, the degrees of relevance set in the relevance degree information are updated every time the operator browses or operates on any piece of diagnosis/treatment data.

The degrees of relevance set in the relevance degree information do not each necessarily have to be the number of times the data has been browsed or operated together with the principal data and may be, for example, an index value defined by the operator.

Further, by referring to the relevance degree information stored in the storage 120, the obtaining function 152 derives a degree of relevance for each piece of diagnosis/treatment data, obtains such diagnosis/treatment data of which the degree of relevance is equal to or higher than the threshold value as the peripheral data from the integrated management DB, and categorizes the peripheral data as the information having a high possibility of being compared with the principal data. In this situation, deriving the degrees of relevance denotes obtaining the degrees of relevance from the relevance degree information.

FIGS. 8 to 16 are drawings for explaining the determining function 153 according to the first embodiment.

In accordance with attributes of the peripheral data with respect to the principal data that were obtained by the obtaining function 152, the determining function 153 is configured to determine a screen configuration indicating positional arrangements of the principal data and the peripheral data to be used at the time of displaying these pieces of data.

More specifically, the determining function 153 is configured to detect the type of the display 140 and to determine the screen configuration in accordance with the type.

For example, the determining function 153 detects whether the display 140 is an HMD or a high resolution display, by using an Application Programming Interface (API) of an Operating System (OS) installed in the medical information processing apparatus 100. Further, the determining function 153 obtains the resolution of the display 140 determined as one of an HMD and a high resolution display. After that, the determining function 153 determines arrangement positions of the principal data and the peripheral data, on the basis of screen configuration information stored in the storage 120.

In this situation, the determining function 153 determines the screen configuration on the basis of the screen configuration information stored in the storage 120.

For example, as illustrated in FIG. 8, the screen configuration information has set therein values of an “application width” and an “application height” indicating the size of an area used for displaying the diagnosis/treatment data and values of a “principal data width” and a “principal data height” indicating the size of an area used for displaying the principal data that are kept in correspondence with information (“4096×2160” and “8192×4320” illustrated in FIG. 8) indicating the resolution levels of the HMD and the high resolution display. In this situation, for the HMD, a field-of-vision space having a spherical shape (such as the shape of an ellipsoid or a perfect sphere) centered on a viewpoint of the operator is defined, so that the size and the position of each of the areas are set on the basis of x-y-z coordinates in the field-of-vision space. For example, a parameter of a spherical function (indicating the curving degree of the display plane) and a definition zone of the x-y-z coordinates, or the like expressing the field-of-vision space may be set.

Further, the determining function 153 determines the screen configuration so that the principal data is arranged in one of the following manners: in a straight-on position of the line of sight of the operator; and at the center of the display screen.

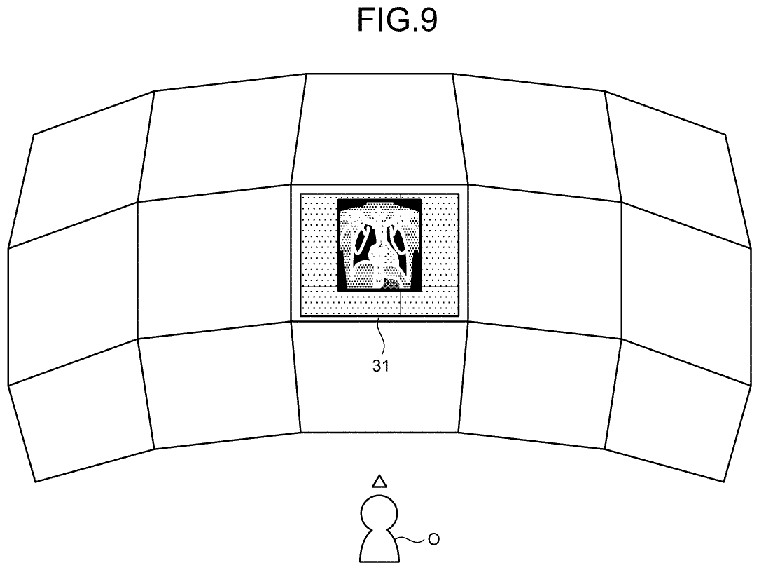

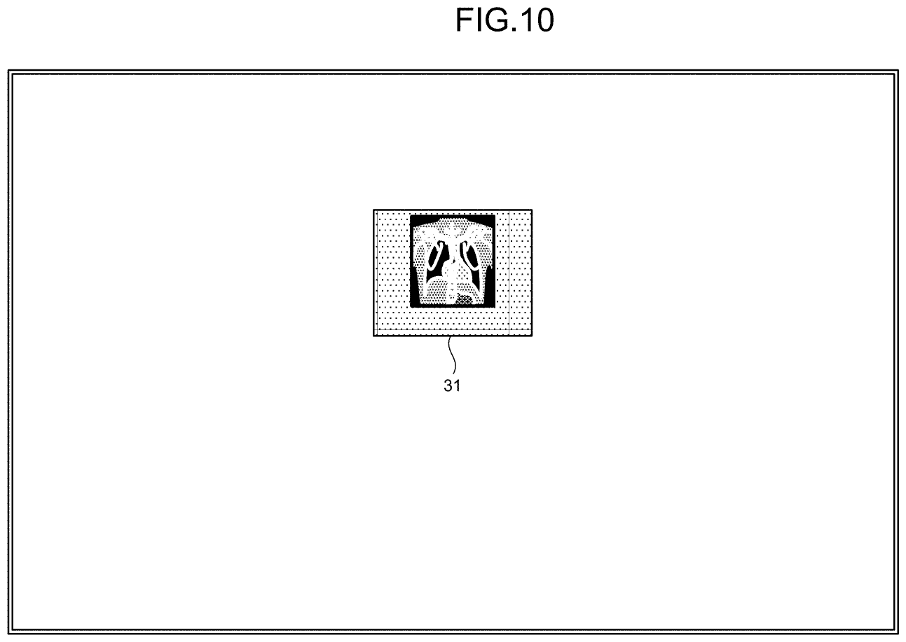

For example, as illustrated in FIG. 9, when the display 140 is an HMD, the determining function 153 arranges the principal data 31 to be in a straight-on position of the line of sight of an operator O. In contrast, for example, as illustrated in FIG. 10, when the display 140 is a high resolution display, the determining function 153 arranges the principal data 31 to be at the center of the display screen. In these situations, in accordance with the type and the resolution of the display 140, the determining function 153 arranges the principal data 31 so as to be contained in the area defined by the “principal data width” and the “principal data height” set in the screen configuration information.

Further, the determining function 153 determines the screen configuration so that the peripheral data categorized as the information of the same type as the principal data is arranged in a time series in a horizontal direction.

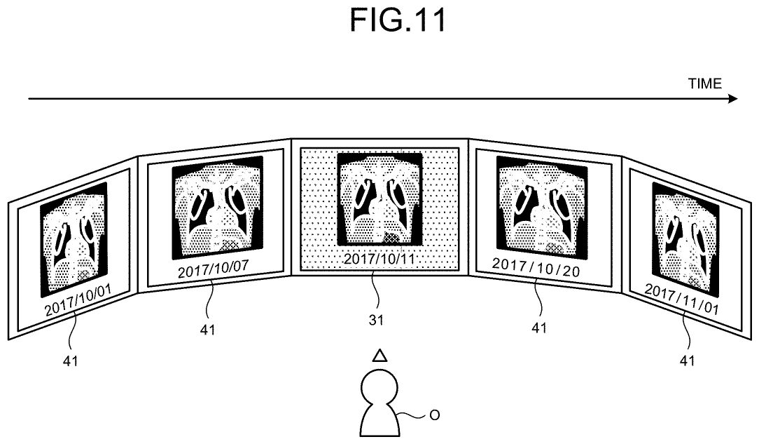

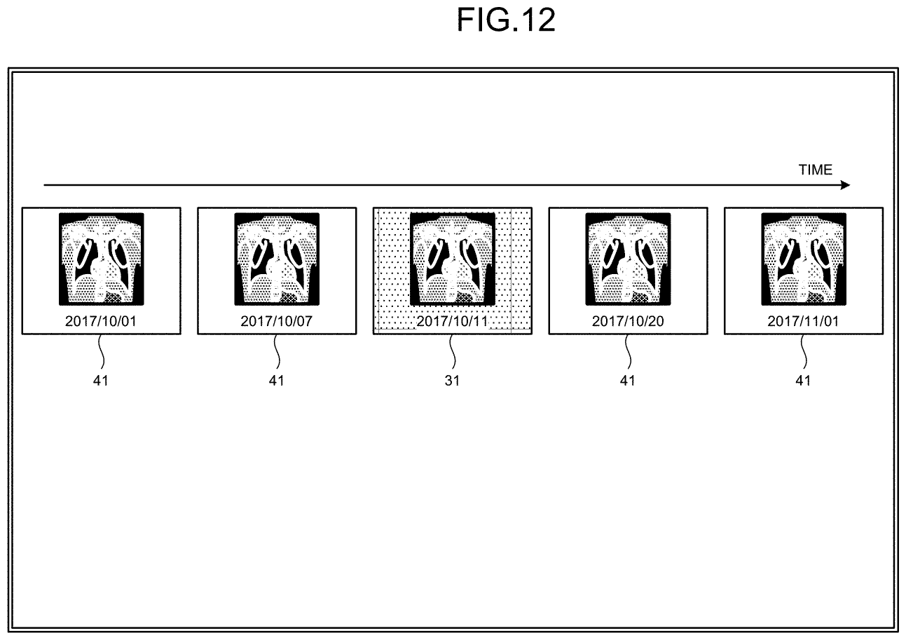

For example, as illustrated in FIG. 11, when the display 140 is an HMD, the determining function 153 arranges pieces of peripheral data 41 categorized as the information of the same type as the principal data 31 to be positioned side by side along a time axis set in the horizontal direction (the left-and-right direction) while being centered on the principal data 31 arranged in the straight-on position of the line of sight of the operator O. In contrast, for example, as illustrated in FIG. 12, when the display 140 is a high resolution display, the determining function 153 arranges the pieces of peripheral data 41 categorized as the information of the same type as the principal data 31 to be positioned side by side on a time axis set in the horizontal direction, while being centered on the principal data 31 positioned at the center of the display screen. In these situations, in accordance with the type and the resolution of the display 140, the determining function 153 arranges the pieces of peripheral data 41 so as to be contained in the area defined by the “application width” and the “application height” set in the screen configuration information.

In this situation, the determining function 153 arranges the pieces of peripheral data 41 acquired earlier than the principal data 31 to be positioned side by side on the left-hand side of the principal data 31, according to the order of acquisition times, starting with the closest one to that of the principal data 31. Further, the determining function 153 arranges the pieces of peripheral data 41 acquired later than the principal data 31 to be positioned side by side on the right-hand side of the principal data 31, according to the order of acquisition times starting with the closest one to that of the principal data 31. Further for example, the determining function 153 displays information indicating the acquisition times of the pieces of data so as to be superimposed on the principal data 31 and the pieces of peripheral data 41 (e.g., “2017/10/01”, “2017/10/07”, “2017/10/11”, “2017/10/20”, and “2017/11/01” in FIGS. 11 and 12).

In this situation, the determining function 153 may arrange the time axis set in the horizontal direction to be on an equal scale or on mutually-different scales between the left-hand side and the right-hand side of the principal data 31. Further, for example, while the pieces of peripheral data 41 are evenly arranged in the horizontal direction, the determining function 153 may set the scale of the time axis to fit the acquisition times of the pieces of peripheral data 41.

Further, the determining function 153 is configured to determine the screen configuration so that the peripheral data categorized as the information explaining the principal data is arranged on the upper side of the principal data.

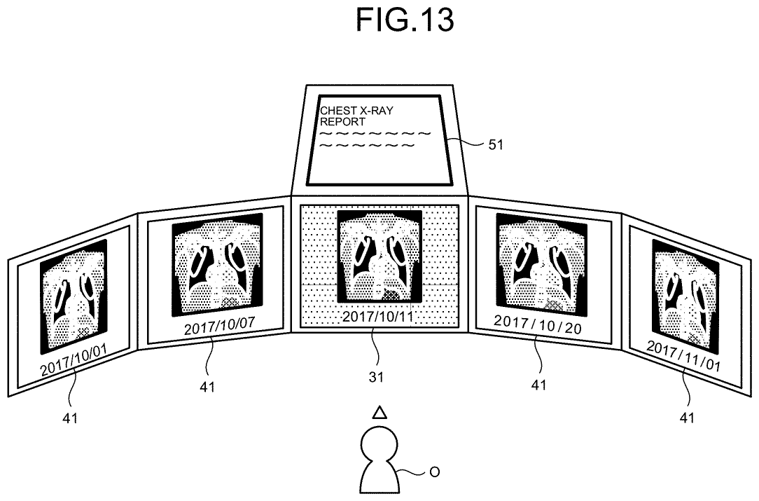

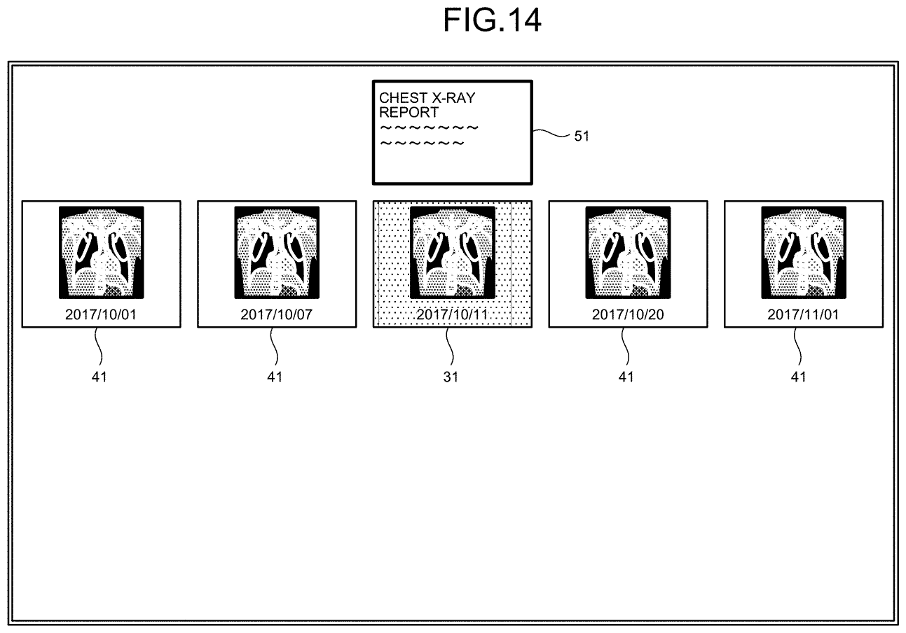

For example, as illustrated in FIG. 13, when the display 140 is an HMD, the determining function 153 arranges peripheral data 51 categorized as the information explaining the principal data 31 to be positioned above the principal data 31 arranged in the straight-on position of the line of sight of the operator O. Alternatively, for example, as illustrated in FIG. 14, when the display 140 is a high resolution display, the determining function 153 arranges the peripheral data 51 categorized as the information explaining the principal data 31 to be positioned above the principal data 31 arranged at the center of the display screen. In these situations, in accordance with the type and the resolution of the display 140, the determining function 153 arranges the peripheral data 51 so as to be contained in the area defined by the “application width” and the “application height” set in the screen configuration information.

In this situation, for example, when there is more than one piece of peripheral data 51 categorized as the information explaining the principal data 31, the determining function 153 arranges the pieces of peripheral data 51 to be positioned side by side on the upper side of the principal data 31, according to the order of levels of detail, starting with the closest one to the level of detail of the principal data 31.

Further, the determining function 153 determines the screen configuration so that the peripheral data categorized as the information having a high possibility of being compared with the principal data is arranged on the lower side of the principal data, in descending order of the degrees of relevance.

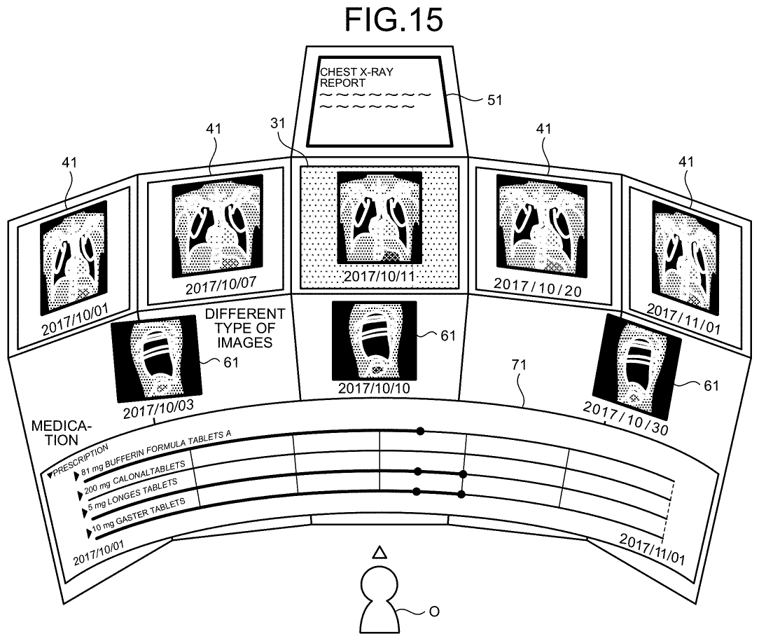

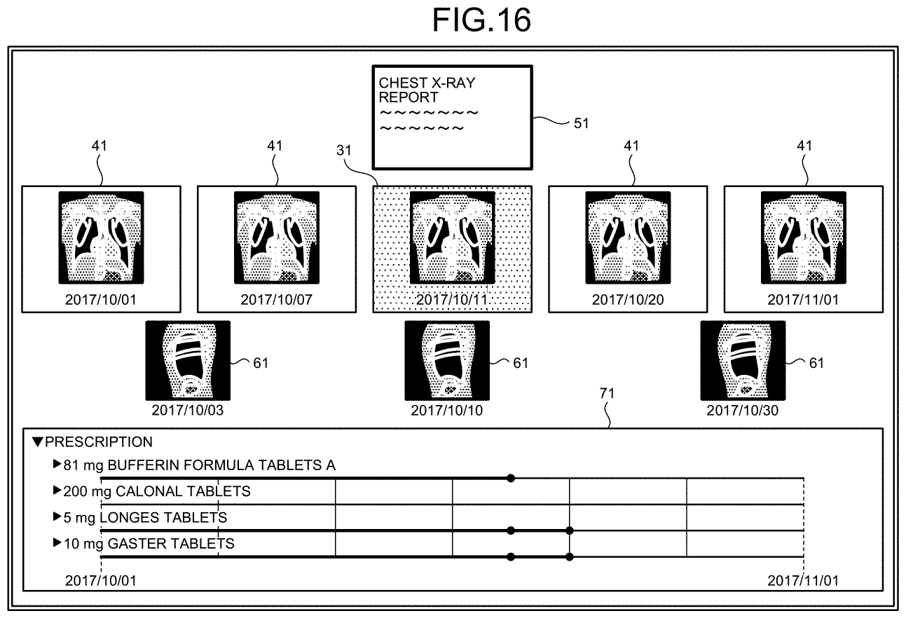

For example, as illustrated in FIG. 15, when the display 140 is an HMD, the determining function 153 arranges different type of image data 61 and medication data 71 serving as peripheral data categorized as the information having a high possibility of being compared with the principal data 31 to be positioned on the lower side of the principal data 31 arranged in the straight-on position of the line of sight of the operator O. Alternatively, for example, as illustrated in FIG. 16, when the display 140 is a high resolution display, the determining function 153 arranges the different type of image data 61 and the medication data 71 serving as the peripheral data categorized as the information having a high possibility of being compared with the principal data 31 to be positioned on the lower side of the principal data 31 arranged at the center of the display screen. In these situations, according to the type and the resolution of the display 140, the determining function 153 arranges the different type of image data 61 and the medication data 71 so as to be contained in the area defined by the “application width” and the “application height” set in the screen configuration information.

In this situation, the determining function 153 arranges the different type of image data 61 and the medication data 71 to be positioned on the lower side of the principal data 31, in the descending order of the degrees of relevance to the principal data 31. For example, when the different type of image data 61 has a higher degree of relevance than the medication data 71 does, the determining function 153 arranges the different type of image data 61 to be positioned below the principal data 31 and arranges the medication data 71 to be positioned further below.

In this situation, for example, when a plurality of pieces of peripheral data having mutually-different acquisition times have been obtained as the pieces of peripheral data categorized as the information having a high possibility of being compared with the principal data 31, the determining function 153 arranges the pieces of peripheral data to be positioned side by side in the horizontal direction along the same time axis as that of the principal data 31. For example, as illustrated in FIGS. 15 and 16, the determining function 153 arranges a plurality of pieces of different types of image data 61 having mutually-different acquisition times to be positioned along the same time axis as that of the principal data 31 and arranges the prescription time period of the medication data 71 to be on the same scale of the time axis as that of the principal data 31.

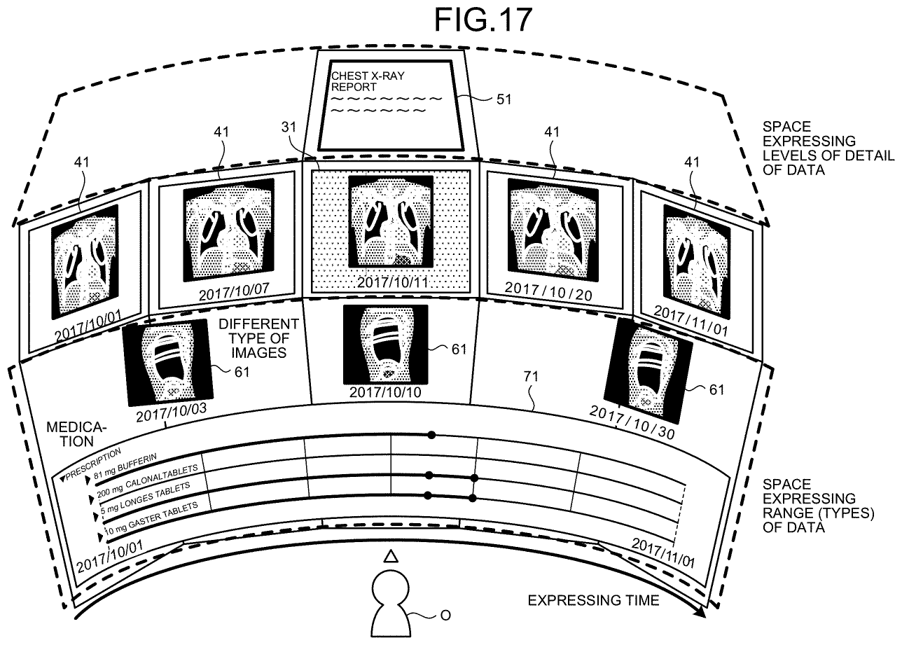

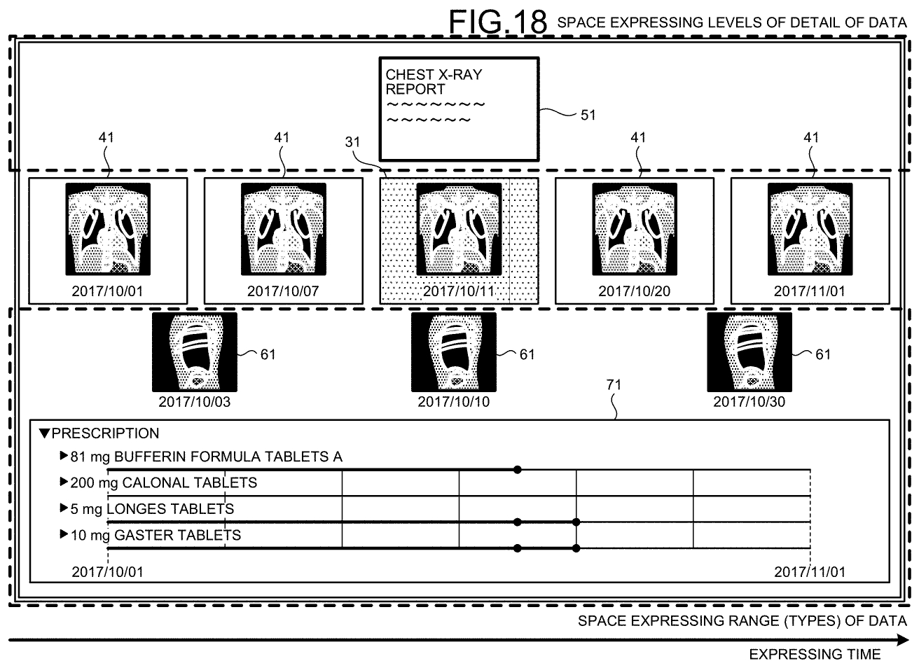

FIGS. 17 and 18 are drawings for explaining the display controlling function 154 according to the first embodiment.

The display controlling function 154 is configured to display the principal data and the peripheral data on the display screen of the display 140 by using the screen configuration determined by the determining function 153.

More specifically, the display controlling function 154 arranges the principal data specified by the specifying function 151 and the peripheral data obtained by the obtaining function 152 to be positioned according to the screen configuration determined by the determining function 153 and to be displayed on the display screen of the display 140.

For example, as illustrated in FIG. 17, when the display 140 is an HMD, the display controlling function 154 displays the principal data 31 in a straight-on position of the line of sight of the operator O. Further, the display controlling function 154 displays the pieces of peripheral data 41 categorized as the information of the same type as the principal data 31, so as to be positioned side by side along the time axis set in the horizontal direction and to be centered on the principal data 31. Further, above the principal data 31, the display controlling function 154 displays the peripheral data 51 categorized as the information explaining the principal data 31. Furthermore, on the lower side of the principal data 31, the display controlling function 154 displays the different type of image data 61 and the medication data 71 serving as the peripheral data categorized as the information having a high possibility of being compared with the principal data 31.

Alternatively, for example, as illustrated in FIG. 18, when the display 140 is a high resolution display, the display controlling function 154 displays the principal data 31 at the center of the display screen. Further, the display controlling function 154 displays the pieces of peripheral data 41 categorized as the information of the same type as the principal data 31, so as to be positioned side by side along the time axis set in the horizontal direction and to be centered on the principal data 31. Further, above the principal data 31, the display controlling function 154 displays the peripheral data 51 categorized as the information explaining the principal data 31. Furthermore, on the lower side of the principal data 31, the display controlling function 154 displays the different type of image data 61 and the medication data 71 serving as the peripheral data categorized as the information having a high possibility of being compared with the principal data 31.

When the display is realized in this manner, the left-and-right direction of the display screen expresses time, while the upper side of the display screen serves as a space expressing the levels of detail of the data, and the lower side of the display screen serves as a space expressing the range (the types) of the data. More specifically, degrees of clinical changes in the diagnosis/treatment data over the course of time is indicated along the horizontal direction of the display screen. Further, levels of clinical details of the diagnosis/treatment data are indicated on the upper side of the display screen. Also, degrees of clinical changes in the other diagnosis/treatment data over the course of time and degrees of effects of diagnosis/treatment actions are indicated on the lower side of the display screen.

The processing functions of the processing circuitry 150 have thus been explained. In the present example, for instance, the processing circuitry 150 is realized by using a processor. In this situation, the processing functions of the processing circuitry 150 are stored in the storage 120 in the form of computer-executable programs. Further, by reading and executing the programs from the storage 120, the processing circuitry 150 realizes the functions corresponding to the programs. In other words, the processing circuitry 150 that has read the programs has the functions indicated within the processing circuitry 150 in FIG. 1. Although FIG. 1 illustrates the example in which the processing functions are realized by the single processor, another arrangement is also acceptable in which processing circuitry is structured by combining together a plurality of independent processors, so that the functions are realized as a result of the processors executing the programs. Further, the processing functions of the processing circuitry 150 may be realized as being distributed among or integrated together in one or more processing circuitries, as appropriate. Further, in the example in FIG. 1, the programs corresponding to the processing functions are stored in the single storage (i.e., the storage 120); however, another arrangement is also acceptable in which a plurality of storages are provided in a distributed manner, so that one or more processing circuitries read corresponding programs from the individual storages.

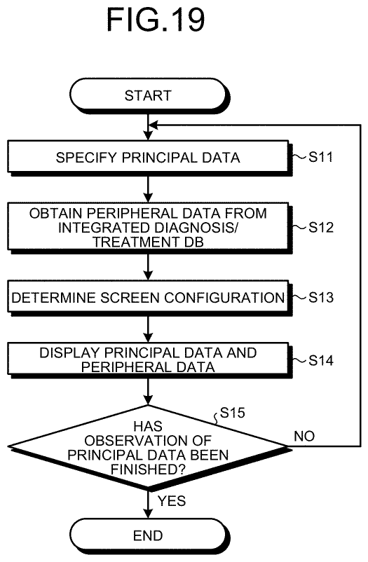

FIG. 19 is a flowchart illustrating a processing procedure of processes realized by the processing functions of the processing circuitry 150 according to the first embodiment.

For example, as illustrated in FIG. 19, in the present embodiment, the specifying function 151 specifies principal data from among the plurality of types of diagnosis/treatment data displayed on the display (step S11). This process is realized, for example, as a result of the processing circuitry 150 reading and executing the predetermined program corresponding to the specifying function 151 from the storage 120.

Subsequently, by using the principal data specified by the specifying function 151 as a reference, the obtaining function 152 obtains peripheral data that is diagnosis/treatment data having a high degree of relevance to the principal data, from the integrated diagnosis/treatment DB structured by the storage 120 (step S12). This process is realized, for example, as a result of the processing circuitry 150 reading and executing the predetermined program corresponding to the obtaining function 152 from the storage 120.

After that, the determining function 153 determines a screen configuration indicating positional arrangements of the principal data and the peripheral data to be used at the time of displaying these pieces of data, in accordance with attributes of the peripheral data with respect to the principal data obtained by the obtaining function 152 (step S13). This process is realized, for example, as a result of the processing circuitry 150 reading and executing the predetermined program corresponding to the determining function 153 from the storage 120.

Subsequently, by using the screen configuration determined by the determining function 153, the display controlling function 154 displays the principal data and the peripheral data on the display screen of the display 140 (step S14). This process is realized, for example, as a result of the processing circuitry 150 reading and executing the predetermined program corresponding to the display controlling function 154 from the storage 120.

Further, for example, unless the operator instructs that observation of the principal data be ended (step S15: No), the processing circuitry 150 exercises control so that the abovementioned series of processes is repeatedly performed. Further, when the operator instructs that the observation of the principal data be ended (step S15: Yes), the processing circuitry 150 ends the processes performed by the processing functions.

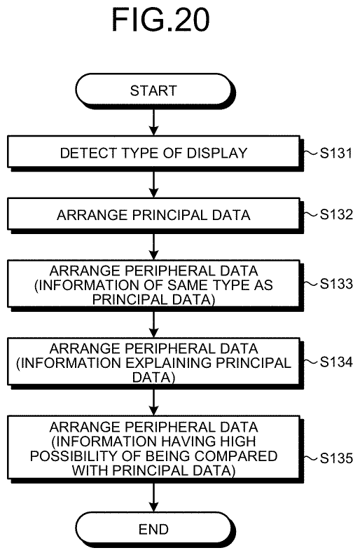

FIG. 20 is a flowchart illustrating a detailed processing procedure of the processes realized by the determining function 153 at step S13 in FIG. 19.

For example, as illustrated in FIG. 20, the determining function 153 detects the type of the display 140 (step S131).

Subsequently, the determining function 153 determines the screen configuration so as to arrange the principal data to be positioned in one of the following manners: in a straight-on position of the line of sight of the operator; and at the center of the display screen (step S132).

After that, the determining function 153 determines the screen configuration so that the peripheral data categorized as the information of the same type as the principal data is arranged in a time series in the horizontal direction (step S133).

Further, the determining function 153 determines the screen configuration so that the peripheral data categorized as the information explaining the principal data is arranged on the upper side of the principal data (step S134).

Furthermore, the determining function 153 determines the screen configuration so that the peripheral data categorized as the information having a high possibility of being compared with the principal data is arranged on the lower side of the principal data in descending order of the degrees of relevance (step S135).

In the present example, the process at step S133, the process at step S134, and the process at step S135 described above may be performed in a different order or may be performed in parallel to one another.

As explained above, in the first embodiment, the specifying function 151 is configured to specify the principal data from among the plurality of types of diagnosis/treatment data. Further, the obtaining function 152 is configured to obtain the peripheral data that is the diagnosis/treatment data having a high degree of relevance to the principal data, by using the principal data specified by the specifying function 151 as a reference. Further, in accordance with the attributes of the peripheral data with respect to the principal data obtained by the obtaining function 152, the determining function 153 is configured to determine the screen configuration indicating the positional arrangements of the principal data and the peripheral data to be used at the time of displaying these pieces of data. Further, the display controlling function 154 is configured to display the principal data and the peripheral data on the display screen of the display 140 by using the screen configuration determined by the determining function 153.

In this configuration, the peripheral data having a high degree of relevance to the specific principal data is displayed on the display screen of the display 140 while being arranged appropriately in accordance with the attribute thereof with respect to the principal data. Consequently, the operator is able to compare the various types of diagnosis/treatment data with one another from multiple aspects.

Further, in the embodiment described above, the determining function 153 is configured to determine the screen configuration so that the principal data is arranged one of the following manners: in a straight-on position of the line of sight of the operator; and at the center of the display screen, while the peripheral data categorized as the information of the same type as the principal data is arranged in a time series in the horizontal direction; the peripheral data categorized as the information explaining the principal data is arranged on the upper side of the principal data; and the peripheral data categorized as the information having a high possibility of being compared with the principal data is arranged on the lower side of the principal data in the descending order of the degrees of relevance.

In this configuration, along the left-and-right direction of the display screen, the degrees of clinical changes in the diagnosis/treatment data over the course of time are indicated. On the upper side of the display screen, the levels of clinical details of the diagnosis/treatment data are indicated. On the lower side of the display screen, the degrees of clinical changes in the other diagnosis/treatment data over the course of time and the degrees of effects of the diagnosis/treatment actions are indicated. Further, the operator is thereby able to compare the mutually-different groups of data in a wide variety with one another, from multiple aspects without the need to adjust the arrangement positions of the pieces of data. Consequently, the operator is able to concentrate on diagnosing processes and treatment plans without having the train of through interrupted.

Further, in the embodiment described above, the determining function 153 is configured to detect the type of the display 140 and to determine the screen configuration in accordance with the type.

With this configuration, by using any of various types of displays such as HMDs and high resolution displays, it is possible to display the principal data and the peripheral data while using an optimal display format for each type. Consequently, it is possible to present the pieces of diagnosis/treatment data more effectively to enable the operator to compare the various types of diagnosis/treatment data with one another from multiple aspects.

For example, possible methods for displaying various types of diagnosis/treatment data on a single screen include a method by which display axes (a diagnosis/treatment data space) are brought into correspondence with pieces of diagnosis/treatment data, so that each piece of diagnosis/treatment data is displayed on an arbitrary one of the axes. According to this method, however, each piece of diagnosis/treatment data would be displayed as being projected onto a two-dimensional space on the arbitrary one of the axes. In contrast, according to the embodiment described above, it is possible to three-dimensionally display the various types of diagnosis/treatment data by using the HMD. The operator is therefore able to compare the various types of diagnosis/treatment data in a more intuitive manner.

Further, for example, as one of the methods for displaying various types of diagnosis/treatment data on a single screen, a method is known by which it is possible to gain a comprehensive understanding of a plurality of pieces of time-series data, by three-dimensionally expressing the time-series data with a perspective projection. According to this method, however, because the diagnosis/treatment data would be displayed along the single time axis on which the present is closer to the viewer and the past is farther from the viewer, it would be impossible, for example, to compare pieces of diagnosis/treatment data at two mutually-different times with each other, while having a comprehensive understanding of the data. In contrast, in the embodiment described above, the various types of diagnosis/treatment data are displayed while being kept in correspondence with one another, according to the levels of detail of the data and the range (the types) of the data, in addition to the time thereof. Consequently, the operator is able to compare the various types of diagnosis/treatment data with one another, while having a more comprehensive understanding of the data.

### Second Embodiment

In the first embodiment described above, for example, another arrangement is also acceptable in which the operator is able to arbitrarily change the amount of the peripheral data to be displayed on the display screen. In the following sections, this example will be explained as a second embodiment. The second embodiment will be explained while a focus is placed on differences from the first embodiment. Detailed explanations of some of the configurations that are the same as those in the first embodiment will be omitted.

In the second embodiment, the determining function 153 is configured to determine a screen configuration so that the amount of the peripheral data changes on the display screen, in response to one of: the operator moving in a depth direction; and an operation performed by the operator.

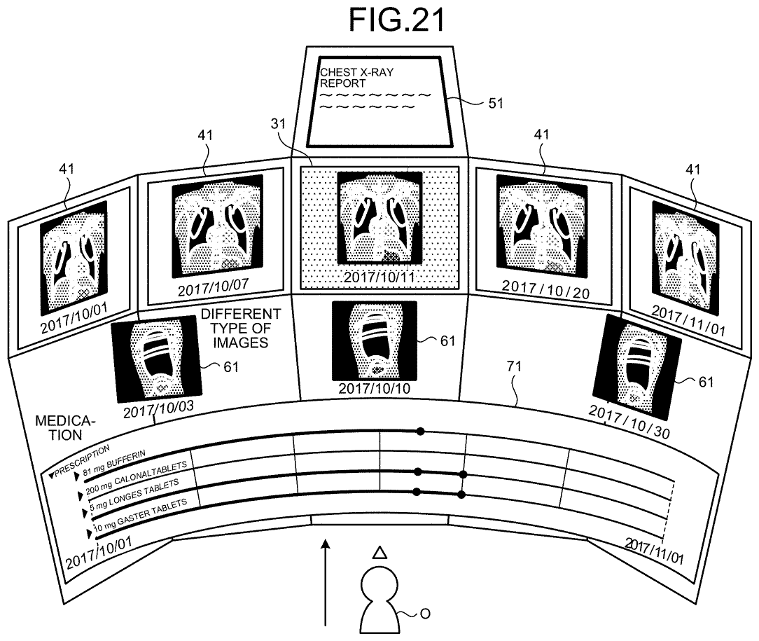

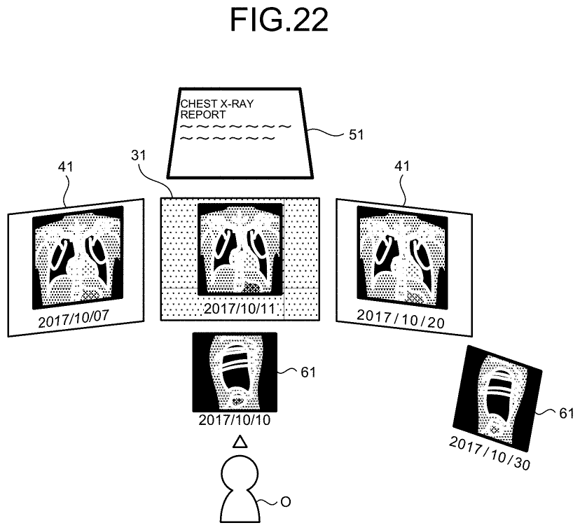

FIGS. 21 and 22 are drawings for explaining the determining function 153 according to the second embodiment.

FIGS. 21 and 22 illustrate an example in which the display 140 is an HMD. The principal data 31 is displayed in a straight-on position of the line of sight of the operator O. Further, the pieces of peripheral data 41 categorized as the information of the same type as the principal data 31 are displayed side by side along the time axis set in the horizontal direction, while being centered on the principal data 31. Further, above the principal data 31, the peripheral data 51 categorized as the information explaining the principal data 31 is displayed. Furthermore, on the lower side of the principal data 31, the different type of image data 61 and the medication data 71 serving as the peripheral data categorized as the information having a high possibility of being compared with the principal data 31 are displayed.

For example, when the display 140 is an HMD, the determining function 153 determines the screen configuration so that the amount of the peripheral data changes on the display screen in response to the operator moving in a depth direction.

For example, as illustrated in FIG. 21, when the operator O moves toward the deeper side, the determining function 153 decreases the amount of the peripheral data, in accordance with the moving amount of the operator O. For example, as illustrated in FIG. 22, with respect to the pieces of peripheral data 41 categorized as the information of the same type as the principal data 31, the determining function 153 sequentially decreases the display of the peripheral data, starting with the peripheral data of which the acquisition time is more distant from that of the principal data 31. Further, with respect to the different type of image data 61 and the medication data 71 serving as the peripheral data categorized as the information having a high possibility of being compared with the principal data 31, the determining function 153 sequentially decreases the display of the peripheral data, starting with the peripheral data of which the degree of relevance to the principal data 31 is lower and of which the acquisition time is more distant from that of the principal data 31. On the contrary, when the operator O moves toward the shallower side, the determining function 153 increases the amount of the peripheral data in accordance with the moving amount of the operator O.

In contrast, when the display 140 is a high resolution display, the determining function 153 determines the screen configuration so that the amount of the peripheral data changes on the display screen, in response to an operation performed by the operator and received via the input interface 130. For example, in accordance with the operation amount of the operation received via the input interface 130, the determining function 153 increases or decreases the amount of the peripheral data, similarly to the example in which the display 140 is an HMD.

As explained above, in the second embodiment, the determining function 153 is configured to determine the screen configuration so that the amount of the peripheral data changes on the display screen of the display 140, in response to one of: the operator moving in a depth direction; and the operation performed by the operator. With this arrangement, the operator is able to arbitrarily switch the display of the diagnosis/treatment data on the display screen between the display in a comprehensive view and the display in a detailed view. Consequently, the operator is able to compare the pieces of diagnosis/treatment data with one another more efficiently.

### Third Embodiment

In the first embodiment, the example was explained in which the obtaining function 152 is configured to derive the degree of relevance for each piece of diagnosis/treatment data by referring to the relevance degree information stored in the storage 120 and to further obtain such diagnosis/treatment data of which the degree of relevance is equal to or higher than the threshold value as the peripheral data categorized as the information having a high possibility of being compared with the principal data; however, possible methods for obtaining the peripheral data is not limited to the method in this example.

For instance, in the example described above, the peripheral data is obtained on the basis of the degree of relevance of each piece of diagnosis/treatment data. Accordingly, depending on the degrees of relevance set in the relevance degree information, there is a possibility that, when the principal data is changed due to moving of the line of sight of the operator or the like, diagnosis/treatment data of a different type from the peripheral data displayed immediately prior may be displayed as peripheral data related to the new principal data.

In this regard, depending on the purpose or the status of use of the medical information processing apparatus 100, when the new principal data and the immediately-prior principal data are diagnosis/treatment data of mutually the same type, there may be some situations where it is desirable to have diagnosis/treatment data of the same type displayed as the peripheral data.

In this situation, examples of the types of diagnosis/treatment data include: diagnosis/treatment data related to an image examination using the same modality; diagnosis/treatment data related to an image examination using another modality; diagnosis/treatment data related to medication administration (diagnosis/treatment data related prescription orders (diagnosis/treatment data related to medication A, diagnosis/treatment data related to medication B, etc.), diagnosis/treatment data related to intravenous drips, diagnosis/treatment data related to injections); and diagnosis/treatment data related to specimen tests.

Accordingly, for example, it is also acceptable to configure the obtaining function 152 to obtain peripheral data by using the type of the principal data as a reference. This example will be explained as a third embodiment. The third embodiment will be explained while a focus is placed on differences from the first embodiment. Detailed explanations of some of the configurations that are the same as those in the first embodiment will be omitted. The third embodiment described below may be carried out in combination with the second embodiment.

More specifically, in the present embodiment, the obtaining function 152 is configured to derive a degree of relevance for each type of diagnosis/treatment data by using the type of the principal data specified by the specifying function 151 as a reference and to further obtain the diagnosis/treatment data of a type having a high degree of relevance, as peripheral data.

For example, when the specifying function 151 has specified new principal data, the obtaining function 152 compares the type of the new principal data with the type of the principal data that was displayed immediately prior and, when the types are the same as each other, derives a degree of relevance to the type of the principal data for each type of diagnosis/treatment data and further obtains such diagnosis/treatment data of the type having a high degree of relevance as peripheral data. In this situation, for example, among the diagnosis/treatment data of the type having a high degree of relevance to the type of the principal data, the obtaining function 152 obtains such diagnosis/treatment data that has a high degree of relevance to the new principal data as the peripheral data.

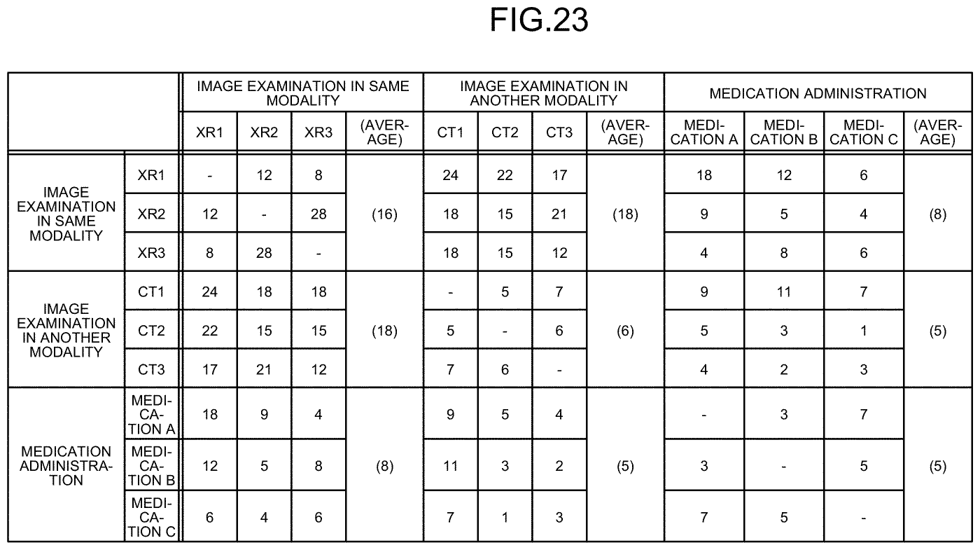

FIG. 23 is a drawing for explaining the obtaining function 152 according to the third embodiment.

In the present embodiment, as the relevance degree information, a degree of relevance to the principal data is set for each piece of diagnosis/treatment data, and further, the storage 120 stores therein information categorizing the principal data and the diagnosis/treatment data according to the types thereof. In other words, in the present embodiment, although in the relevance degree information being used a degree of relevance to the principal data is set for each piece of diagnosis/treatment data similarly to the first embodiment, the relevance degree information is different from that illustrated in FIG. 7 in that the principal data and the diagnosis/treatment data are further categorized according to the types thereof.

In this situation, similarly to the first embodiment, the degree of relevance of each piece of diagnosis/treatment data may be the number of times the piece of diagnosis/treatment data has been browsed or operated together with the principal data or may be an index value defined by the operator. When the degree of relevance indicates the number of times each piece of diagnosis/treatment data has been browsed or operated together with the principal data, the degree of relevance is updated every time the operator browses or operates on the piece of diagnosis/treatment data, similarly to the first embodiment.

For example, as illustrated in FIG. 23, in the relevance degree information, degrees of relevance are set in correspondence with: identification information identifying the types of the principal data (“image examination in the same modality”, “image examination in another modality”, and “medication administration” in the first column from the left in each of the lines in FIG. 23); identification information identifying the principal data (“XR1” to “XR3”, “CT1” to “CT3”, and “Medication A” to “Medication C” in the second column from the left in each of the lines in the table in FIG. 23); identification information identifying the types of the diagnosis/treatment data (“image examination in the same modality”, “image examination in another modality”, and “medication administration” in the first line from the top in each of the columns in the table in FIG. 23); and identification information identifying the diagnosis/treatment data (“XR1” to “XR3”, “CT1” to “CT3”, and “Medication A” to “Medication C” in the second line from the top in each of the columns in the table in FIG. 23).

Further, in the present embodiment, when new principal data has been specified by the specifying function 151, the obtaining function 152, at first, refers to the relevance degree information stored in the storage 120 to specify the type of the new principal data and the type of the principal data that was displayed immediately prior and to compare the types with each other.

In this situation, when the type of the new principal data is the same as the type of the immediately-prior principal data, the obtaining function 152 refers to the relevance degree information and, for each types of diagnosis/treatment data, derives an average value of degrees of relevance of the pieces of diagnosis/treatment data belonging to the type. After that, the obtaining function 152 specifies a type of which the calculated average value of degrees of relevance is the largest and further obtains, from among the pieces of diagnosis/treatment data of the specified type, such pieces of diagnosis/treatment data of which the degree of relevance to the new principal data is equal to or higher than a threshold value as pieces of peripheral data, from the integrated management DB.

In the present example, the average value of the degrees of relevance of the diagnosis/treatment data is used as a degree of relevance between the type of the diagnosis/treatment data and the type of the principal data. Further, the average value of the degrees of relevance of the diagnosis/treatment data may be derived every time the process is performed as described above. Alternatively, the average value may be derived at the time when the operator sets an index value serving as a degree of relevance or at the time when the degree of relevance is updated every time the operator browses or operates on any piece of diagnosis/treatment data, so that the derived average value is registered into the relevance degree information.

In contrast, when the type of the new principal data is different from the type of the immediately-prior principal data, the obtaining function 152 derives a degree of relevance for each piece of diagnosis/treatment data by referring to the relevance degree information and further obtains such diagnosis/treatment data of which the degree of relevance is equal to or higher than a threshold value as peripheral data, similarly to the first embodiment.

With this configuration, in the situation where the principal data has been changed due to moving of the line of sight of the operator or the like, when the new principal data and the immediately-prior principal data are pieces of diagnosis/treatment data of the same type as each other, a diagnosis/treatment data of the same type will be displayed as the peripheral data.

For instance, let us discuss an example in which the relevance degree information is set as illustrated in FIG. 23, and the principal data is changed, due to moving of the line of sight of the operator or the like, from “XR1” to “XR2”, which belongs to the same type as “XR1”, namely “image examination in the same modality”. Also, let us assume that the threshold value used for determining diagnosis/treatment data serving as peripheral data is 20.

In that situation, when the principal data is “XR1”, for example, the diagnosis/treatment data “CT1” (the degree of relevance: 24) and the diagnosis/treatment data “CT2” (the degree of relevance: 22) both of which the degrees of relevance to “XR1” are each equal to or higher than the threshold value are displayed as peripheral data related to “XR1”.

In that state, when the principal data is changed from “XR1” to “XR2”, for example, according to the method explained in the first embodiment, the diagnosis/treatment data “XR3” (the degree of relevance: 28) and the diagnosis/treatment data “CT3” (the degree of relevance: 21) both of which the degrees of relevance to “XR2” are each equal to or higher than the threshold value are displayed as peripheral data related to “XR2”.

In contrast, in the present embodiment, when the principal data is changed from “XR1” to “XR2”, at first, “image examination in another modality” of which the average value (of degrees of relevance) calculated for each type of diagnosis/treatment data is “18”, which is the largest, is specified. Further, among the pieces of diagnosis/treatment data belonging to the specified type “image examination in another modality”, the diagnosis/treatment data “CT3” (the degree of relevance: 21) of which the degree of relevance to “XR2” is equal to or higher than the threshold value is specified and displayed as peripheral data related to “XR2”.

In other words, according to the method described in the first embodiment, when the principal data is changed from “XR1” to “XR2”, not only the diagnosis/treatment data “CT3” which belongs to the same type “image examination in another modality” as “CT1” and “CT2” displayed when the principal data was “XR1”, but also the diagnosis/treatment data “XR3” which belongs to the type “image examination in the same modality” and which was not displayed when the principal data was “XR1” will be displayed as peripheral data related to “XR2”.

In contrast, in the present embodiment, when the principal data is changed from “XR1” to “XR2”, only the diagnosis/treatment data “CT3” which belongs to the same type “image examination in another modality” as “CT1” and “CT2” displayed when the principal data was “XR1” will be displayed as peripheral data related to “XR2”.

As explained above, in the third embodiment, the obtaining function 152 is configured to derive the degree of relevance for each type of diagnosis/treatment data, by using the type of the principal data specified by the specifying function 151 as a reference and to further obtain such diagnosis/treatment data of the type having a high degree of relevance as the peripheral data. With this arrangement, for example, when the new principal data and the immediately-prior principal data are pieces of diagnosis/treatment data of the same type, it is possible to display a piece of diagnosis/treatment data of the same type as the peripheral data. Consequently, it is possible to enhance convenience at the time when the operator compares the various types of diagnosis/treatment data with one another from multiple aspects.

In the embodiments described above, the examples were explained in which an HMD or a high resolution display is used as the display 140; however, possible embodiments are not limited to this example.

For instance, at a conference where a plurality of doctors or the like evaluate diagnoses and/or treatment plans, there may be some situations where both a plurality of personal computers that enable the participants of the conference to view diagnosis/treatment data and a high resolution display that enables all the participants to view the same diagnosis/treatment data are used. In those situations also, the medical information processing apparatus 100 in the present disclosure is applicable.

In those situations, for example, the medical information processing apparatus 100 is connected to the personal computers via the network 200. Further, for example, the display controlling function 154 displays various types of diagnosis/treatment data on a single screen, in response to a request from any of the personal computers. Further, the specifying function 151 detects diagnosis/treatment data focused on by an operator from among a plurality of types of diagnosis/treatment data displayed on the displays of the personal computers and further specifies the detected diagnosis/treatment data as principal data. Further, for each of the personal computers, the obtaining function 152 obtains peripheral data that is diagnosis/treatment data having a high degree of relevance to the principal data, by using the principal data specified by the specifying function 151 as a reference. Further, for each of the personal computers, the determining function 153 determines a screen configuration in accordance with the type of the respective display. Further, for each of the personal computers, the display controlling function 154 displays the principal data and the peripheral data on the display screen of the display, by using the screen configuration determined by the determining function 153.

Further, in that situation, for example, the determining function 153 receives an operation to designate either one of the personal computers or one of the participants, via the input interface 130 or any of the personal computers. After that, when having received the operation, the determining function 153 switches the screen configuration of the principal data and the peripheral data displayed on the display screen of the designated personal computer or participant, to a screen configuration for the high resolution display. Subsequently, by using the screen configuration resulting from the switching by the determining function 153, the display controlling function 154 displays, on the high resolution display, the principal data and the peripheral data displayed on the display screen of the designated personal computer or participant.

In the embodiments described above, the example was explained in which the specifying unit, the obtaining unit, the determining unit, and the display controlling unit of the present disclosure are realized by the specifying function 151, the obtaining function 152, the determining function 153, and the display controlling function 154 included in the processing circuitry 150, respectively; however, possible embodiments are not limited to this example. For instance, instead of being realized by the specifying function 151, the obtaining function 152, the determining function 153, and the display controlling function 154 described in the embodiments, the functions of the specifying unit, the obtaining unit, the determining unit, and the display controlling unit of the present disclosure may be realized by using only hardware or using a combination of hardware and software.

The term “processor” used in the above explanation denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions by reading and executing the programs saved in the storage 120. In this situation, instead of saving the programs in the storage 120, it is also acceptable to directly incorporate the programs in the circuits of the one or more processors. In that situation, the one or more processors realize the functions by reading and executing the programs incorporated in the circuits thereof. Further, the processors in the present embodiments do not each necessarily have to be structured as a single circuit. It is also acceptable to structure one processor by combining together a plurality of independent circuits so as to realize the functions thereof.

In this regard, the programs executed by the one or more processors are each provided as being incorporated in advance in a Read-Only Memory (ROM), a storage, or the like. The programs may be provided as being recorded in a computer-readable storage medium such as a Compact Disk Read-Only Memory (CD-ROM), a Flexible Disk (FD), a Compact Disk Recordable (CD-R), a Digital Versatile Disk (DVD), or the like, in a file in an installable format or in an executable format for the devices. Further, the programs may be stored in a computer connected to a network such as the Internet, so as to be provided or distributed as being downloaded via the network. For example, the programs are structured with modules including the functional units described above. In the actual hardware, as a result of a CPU reading and executing the programs from a storage medium such as a ROM, the modules are loaded into a main storage device so as to be generated in the main storage device.

According to at least one aspect of the embodiments described above, the operator is able to compare the various types of diagnosis/treatment data with one another from multiple aspects.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus, comprising processing circuitry configured:
to specify principal data from among a plurality of types of diagnosis/treatment data;
to obtain peripheral data that is diagnosis/treatment data having a high degree of relevance to the principal data, by using the principal data as a reference;
to determine a screen configuration indicating positional arrangements of the principal data and the peripheral data to be used at a time of displaying the principal data and the peripheral data, in accordance with an attribute of the peripheral data with respect to the principal data; and
to display the principal data and the peripheral data on a display screen of a display by using the screen configuration.

2. The medical information processing apparatus according to claim 1, wherein the processing circuitry derives the degree of relevance for each piece of diagnosis/treatment data by using the principal data as a reference and obtains the diagnosis/treatment data having the high degree of relevance as the peripheral data.

3. The medical information processing apparatus according to claim 2, wherein the processing circuitry derives the degree of relevance on a basis of which of the following the peripheral data is: information of a same type as the principal data; information explaining the principal data; and information having a high possibility of being compared with the principal data.

4. The medical information processing apparatus according to claim 3, wherein the processing circuitry further derives the degree of relevance on a basis of an acquisition time of the peripheral data.

5. The medical information processing apparatus according to claim 1, wherein the processing circuitry detects a type of the display and determines the screen configuration in accordance with the type.

6. The medical information processing apparatus according to claim 1, wherein, on a basis of identification information contained in the diagnosis/treatment data, the processing circuitry categorizes the peripheral data as one of: information of a same type as the principal data; information explaining the principal data; and information having a high possibility of being compared with the principal data.

7. The medical information processing apparatus according to claim 6, further comprising:
a storage configured to store therein relevance degree information in which one selected from between the following is set as the degree of relevance for each piece of diagnosis/treatment data: a number of times the diagnosis/treatment data has been browsed or operated together with the principal data; and an index value defined by an operator, wherein
the processing circuitry derives the degree of relevance for each piece of diagnosis/treatment data by referring to the relevance degree information, further obtains such diagnosis/treatment data of which the degree of relevance is equal to or higher than a threshold value as the peripheral data, and categorizes the peripheral data as the information having the high possibility of being compared with the principal data.

8. The medical information processing apparatus according to claim 6, wherein the processing circuitry determines the screen configuration so that the principal data is arranged in one of the following manners: in a straight-on position of a line of sight of an operator; and at a center of the display screen, and so that the peripheral data categorized as the information of the same type as the principal data is arranged in a time series in a horizontal direction.

9. The medical information processing apparatus according to claim 6, wherein the processing circuitry determines the screen configuration so that the peripheral data categorized as the information explaining the principal data is arranged on an upper side of the principal data.

10. The medical information processing apparatus according to claim 6, wherein the processing circuitry determines the screen configuration so that the peripheral data categorized as the information having the high possibility of being compared with the principal data is arranged on a lower side of the principal data in descending order of the degrees of relevance.

11. The medical information processing apparatus according to claim 1, wherein the processing circuitry determines the screen configuration so that an amount of the peripheral data changes on the display screen in response to one of: an operator moving in a depth direction; and an operation performed by the operator.

12. The medical information processing apparatus according to claim 1, wherein the processing circuitry detects diagnosis/treatment data focused on by an operator from among the plurality of types of diagnosis/treatment data being displayed on the display and further specifies the detected diagnosis/treatment data as the principal data.

13. The medical information processing apparatus according to claim 12, wherein the processing circuitry specifies the principal data on a basis of one of: a line of sight of the operator; and an operation history of the operator.

14. The medical information processing apparatus according to claim 1, wherein
by using a type of the principal data as a reference, the processing circuitry derives a degree of relevance to the type of the principal data for each of the types of diagnosis/treatment data, and
the processing circuitry obtains diagnosis/treatment data of a type having a high degree of relevance, as the peripheral data.

15. The medical information processing apparatus according to claim 14, wherein
when specifying new principal data, the processing circuitry compares a type of the new principal data with a type of principal data displayed immediately prior,
when the types are same as each other, the processing circuitry derives the degree of relevance to the type of the principal data for each of the types of diagnosis/treatment data, and
the processing circuitry obtains diagnosis/treatment data of a type having a high degree of relevance, as the peripheral data.

16. The medical information processing apparatus according to claim 15, wherein, among the diagnosis/treatment data of the type having the high degree of relevance to the type of the principal data, the processing circuitry obtains such diagnosis/treatment data that has a high degree of relevance to the new principal data, as the peripheral data.

17. A medical information processing method, comprising:
specifying principal data from among a plurality of types of diagnosis/treatment data;
obtaining peripheral data that is diagnosis/treatment data having a high degree of relevance to the principal data, by using the principal data as a reference;
determining a screen configuration indicating positional arrangements of the principal data and the peripheral data to be used at a time of displaying the principal data and the peripheral data, in accordance with an attribute of the peripheral data with respect to the principal data; and
displaying the principal data and the peripheral data on a display screen of a display by using the screen configuration.
